(19) United States
(12) Patent Application Publication
(10) Pub. No.: US 2014/0271769 A1
(43) Pub. Date: Sep. 18, 2014

(54) Preparation for the Manufacture of an Implant

(71) Applicant: InnoTERE GmbH, Radebeul (DE)

(72) Inventor: Berthold Nies, Frankisch-Crumbach (DE)

(21) Appl. No.: 14/353,021

(22) PCT Filed: Oct. 19, 2012
(86) PCT No.: PCT/EP2012/070792

§ 371 Date: Apr. 19, 2014

(30) Foreign Application Priority Data
Oct. 19, 2011 (DE) 10 2011 084 801.0

(51) Int. Cl. A61L 27/12; A61L 24/02

(52) U.S. Cl. CPC A61L 27/12; A61L 24/02
USPC 424/422; 424/602

(57) ABSTRACT

The invention relates to a preparation for manufacturing an implant, preferably a bone implant, a process for said manufacture and mouldings obtainable therefrom. The preparation comprises the following components: a) mineral cement powder comprising at least one calcium-ion-containing and/or at least one magnesium-ion-containing inorganic compound as a reactive component; b) at least one organic carrier liquid; c) at least two surfactants selected from at least two of the groups of anionic, cationic, amphoteric and nonionic surfactants; d) less than 1% w/w water based on the total mass of the composition, wherein the weight ratio of the total solids present in the formulation solids to the sum of the weight of the organic carrier liquid and the at least two surfactants is more than if the cement powder contains calcium-ion-containing and no magnesium-ion-containing compounds as the reactive component, or is more than 6 if the cement powder contains magnesium-ion-containing or magnesium-ion- and calcium-ion-containing compounds as the reactive component.

The invention concerns a preparation for the manufacture of an implant, preferably of a bone implant, methods for its manufacture as well as moldings obtainable therefrom. The invention also concerns the use of the preparation as a material for treatment of bone defects or for bone augmentation, for anchoring bone implants or for the manufacture of implantable active ingredient carriers.

Mineral cements, for example, calcium phosphate and magnesium phosphate cements used for the manufacture of implants, are usually provided in powder form. On account of the curing reaction brought on by mixing the powder with water, a solid body is formed. In order to facilitate handling of mineral cements and mixing of the mineral cements with water, preparations are proposed in which mineral cement powders are dispersed in a carrier liquid. The paste available thereby is storage-stable, allows an easy dosage of the mineral cement material, and can be easily mixed with water.

JP 01 139516 A discloses pastes for use as a tooth filler which contain organic carrier liquids and mineral cement powders. Due to the presence of the carrier liquid in the paste, separate mixing with water before use is obsolete. Preferably vegetable oil, polyalcohols, polyglycols, silicone oils, and viscous paraffins are used as carrier liquids. The solids content of the pastes is about 66%. The pastes harden after several hours of contact with water. The moldings obtained by curing of the pastes exhibit a compression strength of maximally about 6 MPa (1 kg/cm² corresponding to 0.1 MPa).

DE 10 2008 028 738 A1 discloses pastes for the manufacture of bone implants in which mineral bone cement powders are contained in one or several anhydrous and water-insoluble carrier liquids. For improving admixture of the solids into the paste and for facilitating admixture of the paste with water, surfactants are preferably added. In spite of the use of anhydrous carrier liquids, introducing the paste into water is enough for initiating the curing reaction. Pastes are described with solids contents of up to 81%, wherein inter alia Tween and Amphisol are added as surfactants. The curing time of the cement preparations amounts to a few minutes. The compression strengths of the moldings obtainable on curing by use of calcium phosphate cements amounted to maximally 14 MPa. For moldings made from magnesium phosphate cements, compression strengths of up to 23 MPa were achieved.

There is still the need for improved cement preparations with which high-strength moldings are obtainable after a minimal curing time. In particular, there is the need for making available cement preparations with resorbable mineral bone cements, in particular calcium phosphate cements, with improved compression strength,

The object of the invention is to provide a preparation for the manufacture of an implant with which calcium ion containing and/or magnesium ion containing implants, in particular calcium phosphate containing implants, with high compression strength can be prepared. Another object of the invention resides in providing a pasty, substantially anhydrous and storage-stable preparation for the manufacture of an implant, which can be caused to undergo curing even without intensive mixing with water, leading to the formation of solid moldings with high compression strength.

The object is solved according to the invention by a preparation for the manufacture of an implant, preferably a bone implant, comprising the following components:
a) mineral cement powder which contains at least one calcium ion containing and/or at least one magnesium ion containing inorganic compound as a reactive component,
b) at least one organic carrier liquid,
c) at least two surfactants selected from at least two of the groups of the anionic, cationic, amphoteric, and non-ionic surfactants,
d) less than 1 wt. % of water relative to the total mass of the preparation,
wherein the weight ratio of the solids contained in total in the preparation to the sum of the weight of the organic carrier liquid and of the at least two surfactants is greater than 5 when the cement powder contains calcium ion containing compounds but contains no magnesium ion containing compounds as a reactive component, with the exception of proportions of magnesium compounds contained as contaminants, or greater than 6 when the cement powder contains magnesium ion containing compounds or magnesium ion and calcium ion containing compounds as a reactive component.

When the cement powder contains calcium ion containing compounds and no magnesium ion containing compounds as a reactive component, this means that no magnesium ion containing compounds are added to this preparation as a reactive component. However, the calcium ion containing compounds may contain customary contaminants or admixtures of magnesium ion containing compounds, i.e. max. 1 wt. %, preferably max. 0.5 wt. %, of magnesium compounds.

According to an advantageous embodiment, the invention comprises a calcium ion containing preparation for the manufacture of an implant, preferably of a bone implant, which comprises the following components:
e) mineral cement powder, preferably mineral bone cement powder, with calcium ion containing inorganic compounds as a reactive component,
f) at least one organic carrier liquid,
g) at least two surfactants selected from at least two of the groups of anionic, cationic, amphoteric, and non-ionic surfactants,
h) less than 1 wt. %, preferably less than 0.1 wt. %, of water (relative to the total mass of the preparation).

In this preparation according to the invention, the weight ratio of the solids contained in total in the preparation to the sum of the weight of the organic carrier liquid and the at least two surfactants is greater than 5. Such a preparation contains no magnesium ion containing compounds as reactive component, except possibly proportions of magnesium compounds contained as contaminants, and is also referred to herein simply as “calcium ion containing preparation”.

According to an advantageous embodiment, the invention comprises a magnesium ion containing preparation for the manufacture of an implant, preferably of a bone implant, which comprises the following components:
a) magnesium ion containing mineral cement powder, preferably magnesium and calcium ion containing mineral cement powder or bone cement powder,
b) at least one organic carrier liquid,
c) at least two surfactants selected from at least two of the groups of the anionic, cationic, amphoteric, and non-ionic surfactants,
d) less than 1 wt. %, preferably less than 0.1 wt. %, of water (relative to the total mass of the preparation).

In such a preparation according to the invention, which contains mineral cement powder with magnesium ion containing or magnesium ion and calcium ion containing compounds as a reactive component, the weight ratio of the solids contained in total in the preparation to the sum of the weight of the organic carrier liquid and the at least two surfactants is greater than 6. Such a preparation is referred to herein also simply as “magnesium ion containing preparation”.

The preparation according to the invention is present in a pasty form. The properties of the paste remain stable for at least 12 months when stored dry. The storage of the preparation has no negative effect on the properties of the molding obtainable after contact with water (comparable properties of the molding). By adjustment of the weight ratio between the solids contained in the preparation and the carrier liquid and the surfactants, it is advantageously possible to provide pasty preparations which produce moldings with high compression strengths of more than 25 MPa, of up to 40 MPa, and in especially preferred formulations of up to 75 MPa, after curing in an aqueous medium. In this context, a weight ratio of solids to the sum of the weight of the carrier liquid and of the at least two surfactants of greater than 5 for calcium ion containing mineral cement powders and a weight ratio of greater than 6 for magnesium ion containing mineral cement powders has been found to be particularly suitable. The preparations according to the inventions are anhydrous, i.e. less than 1 wt. % of water, preferably less than 0 1 wt. % of water, is contained in the preparation.

The compression strength of the moldings obtainable after curing of the preparations according to the invention in an aqueous medium is determined with upright moldings of the dimension 6×6×12 mm along their longest axis with a universal testing machine at a feed rate of 1.0 mm/s. The moldings are produced in that the respective preparations according to the inventions are introduced into molds that are open in upward direction and the latter are then placed into an aqueous 0.9% NaCl solution. The measurement of the compression strength of the moldings occurs after four-day incubation in the NaCl solution, wherein first the molds filled with the preparations according to the inventions are incubated for 24 h at 37° C. and afterwards the removed moldings are incubated for additional 72 h at 37° C. The measurement is carried out immediately after incubation (after approx. 96 h) in the still wet state.

The moldings obtained after curing of the preparations according to the invention achieve a compression strength from 25 to 75 MPa measured in this way.

Preparations according to the invention contain mineral cement powders, preferably mineral bone cement powders. Mineral cement powders mean in the context of the invention mineral solids which react with water under formation of a sparingly soluble solid body. Hydraulically setting mineral cement powders are preferred. Preferably, the mineral cement powder contains silicates, phosphates, sulfates, carbonates, oxides and/or hydroxides, preferably in connection with calcium and/or magnesium ions as a reactive component curable with water. Preferably, the mineral cement powder contains calcium and/or magnesium salts of the ortho-phosphoric acid, the dimeric or polymeric phosphoric acid, glycerophosphoric acid, and further mono-substituted or disubstituted organic phosphoric acid esters, particularly preferred are calcium and/or magnesium salts of the ortho-phosphoric acid. Particularly preferred, the mineral cement powder contains at least one of the following compounds: mono calcium phosphate mono hydrate, mono calcium phosphate anhydrite, dicalcium phosphate anhydrite, dicalcium phosphate dihydrate, octacalcium phosphate, α-tricalcium phosphate, β-tricalcium phosphate, amorphous calcium phosphate, hydroxylapatite, calcium-deficient hydroxylapatite, substituted hydroxylapatite, non-stochiometric hydroxylapatite, nano-hydroxylapatite, tetracalcium phosphate, calcium sulfate, calcium sulfate hemihydrate, calcium sulfate dihydrate, calcium oxide, calcium hydroxide, calcium carbonate, calcium glycerophosphate, calcium citrate, calcium lactate, calcium acetate, calcium tartrate, calcium chloride, calcium silicate, magnesium hydrogen phosphate, trimagnesium phosphate, magnesium dihydrogen phosphate, magnesium chloride, magnesium glycerophosphate, magnesium hydroxide, magnesium hydroxide carbonate, magnesium oxide (MgO), magnesium citrate, calcium magnesium carbonate (dolomite), magnesium silicates.

Preferred calcium ion containing mineral cement powders contain at least one of the following compounds as reactive component: mono calcium phosphate mono hydrate, mono calcium phosphate anhydrite, dicalcium phosphate anhydrite, dicalcium phosphate dihydrate, octacalcium phosphate, α-tricalcium phosphate, β-tricalcium phosphate, amorphous calcium phosphate, hydroxylapatite, calcium deficient hydroxylapatite, substituted hydroxylapatite, non-stochiometric hydroxylapatite, nano-hydroxylapatite, tetracalcium phosphate, calcium sulfate, calcium sulfate hemihydrate, calcium sulfate dihydrate, calcium oxide, calcium hydroxide, calcium carbonate, calcium glycerophosphate, calcium citrate, calcium, lactate, calcium acetate, calcium tartrate, calcium chloride, and calcium silicate.

A magnesium ion containing mineral cement powder in the meaning of the invention is to be understood as a mineral cement powder which contains at least one of the following compounds as a reactive component: magnesium hydrogen phosphate, trimagnesium phosphate, magnesium dihydrogen phosphate, magnesium chloride, magnesium glycerophosphate, magnesium hydroxide, magnesium hydroxide carbonate, magnesium oxide MgO), magnesium citrate or magnesium silicate.

For setting, the magnesium ion containing mineral cement powder furthermore contains hydrogen phosphate, for example, ammonium, sodium or potassium hydrogen phosphate.

Preferably, the mineral cement powder (calcium ion or magnesium ion containing) contains, in addition, strontium compounds, preferably strontium carbonates, strontium oxides, strontium hydroxides and/or strontium phosphates, preferably selected from SrHPO₄, Sr₂P₂O₇, Sr₂(PO₄)₂, and Sr₅(PO₄)₃OH.

The mineral cement powders are preferably ground and fractionated by means of sieving or screening before the addition to a preparation according to the invention.

Powder particles of a size (maximum extension) from 10 tun up to 1,000 μm are used preferably in a preparation according to the invention. Particularly preferred, a preparation according to the invention contains powder particles of the following dimensions:
at least 10 wt. % (relative to the total mass of the contained solids) of a size of less than 5 μm,
at least 25 wt. % of a size of 5 μm to 30 μm, and
at least 20 wt. % of a size of more than 30 μm.

Preferably, a preparation according to the invention contains at least 5 wt. % (relative to the total mass of the contained solids) of powder particles with a size of more than 250 μm. Particularly preferred, the powder particles of a size of more than 250 μm are comprised of a material which is absorbed faster in the body than the mineral cement. Pores are formed in the implant by the resorption of these powder particles in the implant, allowing ingrowth of bone tissue.

Preferably, the mass content of the solids contained in the preparation according to the invention (sum of all solids dispersed and dissolved in the organic carrier liquid with the exception of the at least two surfactants) relative to the total mass of the preparation amounts to more than 75 wt. %, preferably more than 80 wt. %, preferably more than 85 wt. %, particularly preferred more than 87.5 wt. %. Preferably, the mass content of the solids contained in the preparation according to the invention relative to the total mass of the preparation amounts to between 75 and 95 wt. %, preferably between 80 and 95 wt. %, particularly preferred between 85 and 95 wt. %.

Preferably, the mass content of the mineral cement powder contained in the preparation according to the invention relative to the solids contained in total in the preparation according to the invention (sum of all solids dispersed and dissolved in the organic carrier liquid with the exception of the at least two surfactants) amounts to more than 25 wt. %, particularly preferred more than 50 wt. %, further preferred 50 to 80 wt. %.

In this context, the mineral cement powder is the reactive component, the total mass of the solids comprises additionally the contained fillers.

The preparations according to the invention contain at least one organic carrier liquid in which the mineral cement powder is dispersed. The organic carrier liquid is selected such that it itself does not react with the mineral cement powder. In principle, water-soluble as well as sparingly water-soluble carrier liquids are suitable. Sparingly water-soluble in the context of the invention means compounds whose maximum solubility in water amounts to 1.0 mol/l, preferably 0.1 mol/l. Compounds with a maximum solubility in water of more than 1 mol/l (preferably more than 3 mmol/l) are referred to herein as water-soluble. When using water-soluble carrier liquids, a watertight packaging is necessary for the storage of the preparation according to the invention in order to prevent curing of the preparation in ambient air. Hence, sparingly water-soluble carrier liquids are preferred. Particularly preferred are hydrophobic carrier liquids.

Carrier liquids used in preparations according to the invention are preferably bio-compatible.

Preferred sparingly water-soluble carrier liquids are selected from glycerin triacetate, glycerin tributyrate, glycerin trioleate, glycerin dioleate, glycerin monooleate, caprylocaprate, decyl oleate, isopropyl myristate, isopropyl palmitate, oleic acid, oleyl alcohol, oleyl oleate, short chain triglycerides, medium chain triglycerides, short chain and medium chain fatty acid esters of propylene glycol, ethylbenzoyl acetate, ethyl butyrate, ethylbutyryl acetate, ethyl oleate, ethyl caproate, ethyl caprylate, ethyl caprate, ethyl laurate, ethyl levulinate, ethyl myristate, ethyl palmitate, ethyl linoleate, ethyl stearate, ricinoleic acid, linoleic acid, linolenic acid, arachidic acid, oleinic acid, ethyl arachidate, α-tocopherol, β-tocopherol, γ-tocopherol, δ-tocopherol, benzyl alcohol, benzyl benzoate, diethylbutyl malonate, diethylene glycol dibutylether, diethyl ethyl malonate, diethylphenyl malonate, diethyl phthalate, diethyl sebacate, diethyl suberate, diethyl succinate, dibutyl maleinate, dibutyl phthalate, lecithin, paraffin oil, petrolatum, liquid paraffins, and esters of sebacic acid. Particularly preferred carrier liquids are short or medium chain triglycerides or medium chain fatty acid esters of ethylene glycol and propylene glycol. Short chain fatty acid compounds are understood as compounds of fatty acids of a length of 2 to 5 carbon atoms each. Medium chain fatty acid compounds are understood as compounds of a length of 6 to 14 carbon atoms each.

Particularly preferred sparingly water-soluble carrier liquids are selected from esters of fatty acids and monovalent or multivalent alcohols. Of these, triglycerides are preferred. Of these, particularly preferred are triglycerides with contained fatty acids which have on average fewer than 14 C atoms. Further preferred sparingly water-soluble carrier liquids are polypropylene glycols and esters of polypropylene glycols as well as mono ethers and diethers of polypropylene glycols with mono alcohols.

Preferred water-soluble carrier liquids are selected from polymers of ethylene glycol, short chain oligomers of propylene glycol, co-polymers with ethylene glycol and propylene glycol units, mono and dimethyl ethers of polyethylene glycol, glycerin, and its water-soluble ethers and esters and di-glycerin and polyglycerin.

Preferred preparations according to the invention contain at least one water-soluble and at least one sparingly water-soluble organic carrier liquid. Thereby, the setting rate of the preparation according to the invention in water can be influenced advantageously. Another advantage of the combination of at least one water-soluble and at least one sparingly water-soluble carrier liquid is that the solids content of the paste can be increased.

In principle, the organic carrier liquid can also be a (preferably non-ionic) surfactant which is existing in liquid physical state. Nevertheless, in addition to the at least two surfactants, an organic liquid which is not a surfactant is preferably included as a carrier liquid in a preparation according to the invention.

Preferably, the weight proportion of the organic carrier liquid relative to the total mass of the preparation amounts to 5 to 25 wt. %, preferably 5 to 20 wt. %, preferably 5 to 15 wt. %, preferably 5 to 12.5 wt. %.

Preferably, a preparation according to the invention contains furthermore at least one setting accelerator. In this way, the setting time and pH value course are advantageously set during curing of the preparation according to the invention. Preferred setting accelerators are phosphate salts, organic acids or salts of organic acids. Sodium and/or potassium ion containing phosphates or sodium and/or potassium ion containing salts of organic acids are preferred. Particularly preferred are potassium ion containing phosphates (preferably potassium phosphates, in particular potassium dihydrogen phosphate and di-potassium hydrogen phosphate). With potassium ion containing phosphates, especially advantageous setting kinetics has been achieved, particularly in combination with non-ionic surfactants (particularly well in combination with non-ionic and anionic surfactants). A particularly preferred preparation according to the invention contains at least one ionic (anionic or cationic) and at least one non-ionic surfactant with a medium chain triglyceride as an organic carrier liquid as well as at least one potassium ion containing phosphate (preferably di-potassium hydrogen phosphate) as a setting accelerator. A long durability of the preparation is advantageously achieved thereby.

The setting accelerator is preferably contained in a mass proportion (relative to the mass of the mineral cement powder) of 0.1 to 5%, particularly prefers 0.2 to 4%, particularly preferred 0.5-3.5%, in the preparation according to the invention.

The preparations according to the invention contain at least two different surfactants, selected from at least two of the groups of anionic, cationic, amphoteric, and non-ionic surfactants. Preferably, at least one non-ionic surfactant is contained. Preferably, at least one anionic surfactant is contained. Particularly preferred, at least one non-ionic and at least one anionic surfactant are contained in a preparation according to the invention and the total mass of the non-ionic surfactants is preferably at least double that of the total mass of the anionic surfactants.

The surfactants facilitate the incorporation of the solids into the organic carrier liquid.

Particularly preferred are combinations of at least one hydrophilic surfactant and at least one lipophilic surfactant. Most particularly preferred, at least one surfactant with an HLB value (hydophilic lipophilic balance, mass ratio between the polar and the non-polar part of a surfactant) of more than 8 (hydrophilic surfactant) and at least one surfactant with an HLB value of less than 5 (lipophilic surfactant) are contained in a preparation according to the invention.

Surfactant are preferably contained in a preparation according to the invention in a mass proportion in total (sum of the mass of all contained surfactants relative to the total mass of the preparation) of 0.1 to 10 wt. %, preferably 0.5 wt. % to 5 wt. %, preferably 1 wt. % to 3.5 wt. %.

When at least one non-ionic surfactant is contained in a preparation according to the invention, the non-ionic surfactant (total mass of the non-ionic surfactants) and the organic carrier liquid are present in a weight ratio of 1:1 to 1:25, preferably 1:5 to 1:20.

When anionic and non-ionic surfactants are contained in a preparation according to the invention, the total mass of the non-ionic surfactants preferably is at least double the total mass of the anionic surfactants

Preferred non-ionic surfactants are selected from fatty alcohols (preferably decyl alcohol or dodecyl alcohol), ethoxylated fatty alcohols (preferably CH₃(CH₂)ₓ—O—(CH₂CH₂O)_{y}—H with x=8-18 and Y=2-300), ethylene oxide/propylene oxide block copolymers, alkylphenol ethoxylates, alkyl polyglucosides, ethoxylated fats and oils, alkanol amides, ethoxylated alkanol amides, polyethylene glycol fatty acid esters, glycol and glycol esters (preferably ethylene glycol fatty acid esters, propylene glycol fatty acid esters or glycerin fatty acid esters), sorbitan esters (preferably mono and triesters), sugar esters, ester/ether surfactants (preferably ethoxylated glycol and glycerin esters), ethoxylated sorbitan esters (preferably ethoxylated sorbitan esters of fatty acids laurinic, myristic, palmitic, stearic, and oleic acid), polyglycerin monoesters, and amine oxides.

Preferred anionic surfactant are selected from fatty acids and their salts (preferably sodium, potassium, ammonium, calcium, magnesium, zinc, iron salts, particularly preferred sodium oleate, sodium palmitate), esters of fatty acids and their salts (preferably sodium dilaureth-7-citrate, stearoyl disodium tartrate), carboxylic acid ethers (preferably fatty alcohol polyglycol ether carboxylic acid), alkyl sulfates (preferably sodium alkyl sulfate, particularly preferred sodium lauryl sulfate), alkylether sulfates, alkyl sulfonates (preferably sodium lauryl sulfonate), sulfosuccinates (preferably sodium dialkyl sulfosuccinate), phosphoric acid esters and salts thereof (preferably alkyl and alkylether phosphates), citric acid esters of mono and diglycerides, acylamino acids and salts thereof (preferably acyl glutamates, acyl peptides, acyl sarcosides).

Particularly preferred, a preparation according to the invention contains at least one aforementioned anionic and at least one aforementioned non-ionic surfactant, particularly preferred at least
one ethoxylated fatty alcohol, at least one ethoxylated fatty acid, at least one ethoxylated sorbitan fatty acid ester (in particular polysorbate 80), at least one ethoxylated fat or at least one ethoxylated oil (in particular polyethoxylated castor oil) and
at least one fatty alcohol esterified with citric acid, sulfuric acid or phosphorus acid, at least one mono or diglyceride esterified with citric acid, sulfuric acid or phosphoric acid or salts thereof, or at least one fatty acid esterified with citric acid, sulfuric acid or phosphoric acid or salts thereof.

Preferably, preparations according to the invention contain at least one filler. Filers are understood in the meaning of the invention as substances which are not already contained in the mineral cement powder when preparing the preparation according to the invention, but are added only after dispersion of the mineral cement powder in the organic carrier liquid in the presence of the at least two surfactants. The fillers are added in this context for adjusting the properties (in particular flowability, resorbability, or X-ray contrast) of the preparation according to the invention. Preferred fillers are selected from strontium carbonate, strontium hydrogen phosphate, strontium phosphate, glass ceramics (preferably resorbable glass ceramics, in particular glass ceramics comprising SiO₂, Na₂O, CaO and P₂O₅ and optionally containing in addition MgO and/or K₂O), calcium carbonate, iron oxides, silicon dioxides, barium sulfate, glycerin stearate, precipitated nanocrystalline hydroxylapatite, calcium deficient hydroxylapatite and tricalcium phosphate, in particular beta-tricalcium phosphate. The fillers are present in particulate form.

As fillers, mineral or organic fibers are also advantageously suitable. Examples are organic fibers on the basis of resorbable polymers which are derived, e.g., from the resorbable suture materials. Such fibers can raise the breaking strength of the cured cement. Conventional mineral bone cements with contained polymeric short fibers are known from the literature (in particular Norian screwable).

Preparations according to the inventions which contain short mineral fibers as fillers, in particular ceramic and glassy short fibers, are preferred. Examples of such fibers are fibers on the basis of wollastonite and (in particular bio-soluble) glass fibers, comprising also silica gel fibers and silicic acid fibers. Examples of suitable glass fibers are in particular short staple fibers on the basis of lime alkali glasses, so-called bioglasses, in particular bioglass of the composition 45S5 (45S5 indicates with “45” the percentage of SiO₂ and with “5” the ratio of CaO to P₂O₅). Numerous other formulations are described in the scientific literature.

Silicic acid fibers, e.g., Belcotex fibers of the company Belchem, Freiberg, with average fiber diameters<10 μm and fiber lengths of <5 mm are preferred also. Particularly preferred are fibers without aluminum proportion, in particular those which contain, in addition to SiO₂ and optionally phosphate, exclusively alkali and earth alkali ions.

The mineral fibers are contained in preparations according to the invention in a quantity of 0.1 to 30 wt. %, preferably between 1 and 20 wt. %. They have a great effect on the breaking behavior of the cured moldings as can be taken from the results of Example 6. While the compression strength rises only slightly, the reinforced moldings do not break catastrophically, but can still absorb load even after surpassing the maximum compressive force. This deformation behavior is especially advantageous for implant applications because a partially broken implant can be integrated again by the regeneration process in the bone.

Preferred fibers have an average diameter of 1 μm to 300 μm, preferably 3 μm to 100 μm.

Substances which react with water under formation of a sparingly soluble solid body (and thus have the identical chemical composition as mineral cement powders in the meaning of the invention) can be contained as a filler in the preparation according to the invention, in addition to the contained mineral cement powder. For providing the mineral cement powder, its components are mixed with each other under grinding action. The fillers which are added to the preparation according to the invention are not ground together with the components of the mineral cement powder. Fillers are added to the preparation according to the invention only after mixing of the mineral cement powder with the organic carrier liquid. In this manner, due to the composition of the particle dimensions (the particle size is to be understood herein as the maximum extension of the particles, in case of round particles this corresponds to the particle diameter) of the fillers, inter alia the rheologic properties of the preparation according to the invention are influenced. In addition, the mechanical properties of the preparations according to the invention and of the moldings obtainable therefrom can be influenced by adding fillers.

Preferably, preparations according to the invention contain more than 5 wt. % (relative to the total mass of the solids contained in the preparation) particulate fillers with a particle size of less than 10 μm. These are selected preferably from strontium carbonate, calcium carbonate, precipitated nanocrystalline hydroxylapatite, and calcium deficient hydroxylapatite.

Preferably, preparations according to the invention contain more than 20 wt. % of particulate fillers with a particle size of more than 50 μm, preferably more than 100 μm. In this context, the particle size of the fillers amounts to preferably maximum 5 mm. Fillers with a particle size of more than 50 μm are selected preferably from α-tricalcium phosphate, β-tricalcium phosphate, calcium hydrogen phosphate, glass ceramics (preferably resorbable glass ceramics, in particular glass ceramics comprising SiO₂, Na₂O, CaO, and P₂O₅ and optionally, in addition, containing MgO and/or K₂O), sintered hydroxylapatite, calcium carbonate, sintered or fired magnesium phosphate, calcium magnesium phosphate, magnesium ammonium phosphate (as a hydrate or anhydrous).

Preferably, the preparations according to the invention contain at least one polymeric auxiliary, preferably selected from collagen, gelatin and their derivatives, starch and its derivatives (preferably hydroxyethyl starch, carboxymethyl starch), cellulose derivates, chitin, chitosan and their derivatives, polyvinyl alcohol, polyvinyl pyrrolidone, polyacrylic acid and its derivatives (in particular polycarboxylate ether), polymethacrylic acid and its derivatives (in particular copolymers of methacrylic acid with methylmethacrylate, ethylacrylate and/or methylacrylate), polymethylmethacrylate, polystyrene, and copolymers with monomers of methylmethacrylate and styrene.

Preferably, the preparations according to the invention contain water-soluble particulate fillers of mineral or organic substances. The porosity of the solid material that is formed by curing with water can be adjusted advantageously by using water-soluble particles. Water-soluble fillers have preferably a particle size of 50 μm up to 2,000 μm, preferably of 100 μm up to 1,000 μm. Water-soluble fillers are contained preferably in the preparation according to the invention in 5 to 90 vol. %, preferably 10 to 80 vol. % (relative to the total volume of the preparation according to the invention). Preferred water-soluble fillers are selected from sugars (preferably saccharose), sugar alcohols (preferably sorbitol, xylitol, mannitol), water-soluble salts (preferably sodium chloride, sodium carbonate or calcium chloride). The water-soluble fillers are preferably used in the form of granular materials.

Preferably, preparations according to the invention contain at least one pharmaceutically active agent, preferably active agents with growth-stimulating or anti-microbial action. Preferred active agents are selected from antibiotics, antiseptics, antimicrobial peptides, antiresorptive agents (preferably bisphosphonates, corticoids, fluorides, proton pump inhibitors), bone growth stimulating agents (preferably growth factors, vitamins, hormones, morphogenic agents, of these preferably bone-morphogenic proteins).

The preparations according to the invention are formulated preferably as a single paste system. According to one embodiment, the preparations according to the invention are formulated as a two-component system with an anhydrous paste and a water-containing component.

The anhydrous paste contains for this purpose the preparation according to the invention in accordance with one of the embodiments described above.

The water-containing component contains for this purpose an aqueous solution, an aqueous dispersion or pure water.

Surprisingly, it was found that the preparations according to the invention can be mixed very well and macroscopically homogeneously with a very broad spectrum of water-containing components. An excellent mixing is also achieved when the water-containing component is existing as a solution of water-soluble polymers, dissolved active agents such as, for example, antibiotics, or as an aqueous dispersion of solids such as for example, dispersed bone minerals or their synthetic analogs. Also very good mixing was achieved when as a water-containing component blood, blood serum, bone marrow aspirate or platelet-rich plasma was used. Accordingly, the preparation according to the invention can be combined variably. The combination of the preparation according to the invention with an aqueous solution or dispersion is especially advantageous when using a double chamber cartridge with a fixedly predetermined mixing ratio of the two components to be mixed, wherein mixing occurs upon discharge. The mixture of the preparation according to the invention is preferably as follows: aqueous component in a ratio of ≧2:1 to 4:1, particularly preferred in a ratio of ≧4:1 to 10:1.

Advantages of the two-component system are an easy admixture of water-soluble active agents and biological components which are to be combined only on site with the preparation according to the invention, furthermore avoidance of incompatibilities during the preparation and storage, and control of the setting conditions by metered addition of the aqueous component. With large amounts of implanted material, mixing with an aqueous component is more reliable and leads to quicker setting compared to passive curing with a single-paste system.

Also encompassed by the invention is the use of a preparation according to the invention for manufacturing a material for the treatment of bone defects or bone augmentation, for anchoring bone implants or for manufacturing implantable active agent carriers. The compression strength of more than 25 MPa is advantageous in this context.

The invention also encompasses methods for manufacturing an implant, preferably a bone implant. In this context, a preparation according to the invention is contacted with a water containing preparation. A setting reaction is brought on by the reaction of the water with the mineral cement powder and a solid body is formed. The water containing preparation is in this context either pure water, an aqueous solution or a dispersion of solids in an aqueous solvent (aqueous paste). For initiating the setting reaction the introduction (insertion) of the preparation according to the invention into water or an aqueous solution is enough. The initiation of the setting reaction of a preparation according to the invention can also occur after implantation wherein the preparation according to the invention cures due to the water that is available in the environment of the implant. When an aqueous paste is used as a water containing preparation, the preparation according to the invention is mixed with the paste (preferably with stirring) and thereby the mineral cement components are contacted with the water. This occurs before implantation in the body. For this purpose, the preparation according to the invention is present preferably in a suitable mixing system, in particular a two-chamber syringe, wherein in one chamber the preparation according to the invention and in the other chamber the water containing preparation is contained.

Three-dimensional moldings are produced preferably from a preparation according to the invention. On account of the high solids content of the preparation according to the invention, moldings of the preparation according to the inventions are also stable in the uncured state after packaging for long periods of time without change of the dimensions (collapsing).

Due to the advantageous flow properties of the preparation, it is suited for use in methods of three-dimensional plotting (3D plotting). In this context, a three-dimensional molding is constructed by extrusion. Here it is also especially advantageous that the preparation according to the invention can be metered well (high structural viscosity) and at the same time has a high solids content so that the moldings do not collapse even in the uncured state to thereby change the previously defined dimensions. After introducing the three-dimensional molding into water or into an aqueous solution, the molding cures. In this manner it is advantageously possible to provide an implant that is tailor-made for the patient.

The invention also comprises solid moldings which are obtainable after contacting a preparation according to the invention with a water containing preparation (preferably water, an aqueous solution or a dispersion of solids in an aqueous solvent). The moldings are preferably of an open pore structure and have an interconnected pore system. In this context, the pores have preferably an average diameter of >50 μm and <1,000 μm (in this context, the average diameter is the maximum extension that is averaged across all pores of the pores of the entire molding). It is advantageously possible with the method of 3D plotting to adjust the pore system of the molding in a targeted fashion and to alternatively generate uniform pore systems or to arrange pores in preferred directions in this way.

Preferably, the moldings which have pores with a maximum diameter of 50 μm (solid moldings that are free of macropores, they are obtainable by curing a preparation according to the invention in aqueous environment), exhibit a compression strength of more than 20 MPa. The molding which is formed by curing a preparation according to the invention shows preferably a Ca/PO₄ ratio of more than 1.35, preferably at least 1.5, preferably at least 1.6.

The invention also comprises a method for manufacturing a preparation according to the invention. In the method
a) mineral cement powder or individual components of the mineral cement powder are dispersed successively by grinding in the organic carrier liquid in the presence of at least two surfactants.
b) Preferably, afterwards further components of the mineral cement powder and/or fillers, in each case with a particle size of less than 10 μm, are added with continuous grinding.
c) Afterwards, fillers with a particle size of more than 50 μm are added preferably. The preparation is mixed further (preferably by stirring or kneading) until a homogeneous paste is formed. The method step c) is carried out preferably without grinding.

With this method, it is advantageously possible to prepare preparations according to the invention with solids content of more than 85 wt. % (relative to the total mass of the preparation). In this context, in the first method step, the components of the mineral cement powder that are reactive with water are first ground and dispersed in the organic carrier liquid in the presence of at least two surfactants. The mineral cement powders are produced for this purpose beforehand with known methods, preferably by grinding of the contained components that are reactive with water. In a second method step, preferably very fine particulate particles with a maximum extension of less than 10 μm (e.g., as crystallization seeds) are added with grinding action so that the viscosity of the preparation is greatly increased. In a third method step, the solids content of the preparation is primarily adjusted by the addition of large particles with a maximum extension of more than 50 μm. For this reason, no grinding process is thus used for the addition of the coarse particles, but the particles are merely admixed to the preparation formed in the first or second method step.

In the method, first the surfactants are added in the first method step to the carrier liquid until a homogeneous liquid is formed. Afterwards, the mineral cement powder or its components are added in portions with grinding action until a homogeneous paste has formed.

With the invention, preparations for the manufacture of implants are provided which can be processed easily and are storage-stable and with which implants with high compression strengths can be produced. On account of water being essentially absent in the preparation, it cures only after introducing it into water or an aqueous solution. By using several surfactants, the incorporation of solids into the organic carrier liquid is facilitated.

On account of the use of an especially high weight ratio of the solids contained in total in the preparation relative to the sum of the weight of the organic carrier liquid and the at least two surfactants of greater than 5 (for calcium ion containing mineral cement powders) and of greater than 6 (for magnesium-containing mineral cement powders), moldings (implants) with an especially high compression strength are advantageously obtainable. It has be found surprisingly that the compression strength of the obtainable moldings increases suddenly upon surpassing the aforementioned weight ratios in the preparation.

When considering the content of dispersed solids, it must be taken into account that an increase of the solids proportion in a dispersion can have a very big effect even for nominally small differences. An increase of e.g. 80% to 85% of solids content means that 25% less liquid is available for dispersion of the solids (relative to 100 g: 15 g of carrier liquid instead of 20 g). This is even more relevant since a portion of the carrier liquid consists of surfactants which adhere as surface-active substances to the mineral particles and are therefore available only to a limited extent as a component of the carrier liquid. Also, Amphisol A (in the examples) is moreover a solid which is dissolves only upon heating in the carrier liquid and precipitates again upon cooling. Hence, the described increase of the solids content is thus to be considered a significant change of the composition in comparison to the prior art.

The highest solids content for single-component calcium phosphate cement pastes described in the prior art is listed in Table 1 as a comparative example For a total solids content of 81.2% and a corresponding weight ratio of solid to carrier liquid of 4.32:1, the cement of the comparative example is comparable in its consistency and in its processing properties to the preparations 4 and 5 according to the invention in Table 1. However, the preparations 4 and 5 according to the invention in Table 1 have in contrast to the comparative example a solids content of 87.84% in each case, according to a weight ratio of solid to carrier liquid of 7.22:1. This greatly increased ratio of solid to carrier liquid causes, at comparable paste consistency, a significant improvement of the paste stability with respect to sedimentation or separation and a duplication of the compression strength of the test bodies made from these preparations.

Surprisingly, it was also found that the preparations according to the invention—in spite of the high loading with solids and the intensive grinding/mixing which leads to a very dense and practically pore-free dispersion—spontaneously cure after introduction into an aqueous solution (in the standard tests an 0.9% saline solution was used). In the corresponding tests, the produced pasty preparations according to the invention were filled, without further mixing with water or aqueous solutions, into parallelepipedal molds of the dimension 6×6×12 mm such that the respective mold was filled completely. Afterwards, the filled molds were immersed into the 0.9% saline solution and incubated at 37° C. for 24 hours. After this time, the paste had cured to moldings and the moldings were removed as solid blocks. The removed moldings were incubated further for additional and complete curing for additional 72 hours at 37° C. The compression strength tests were carried out with moldings made in this way. The measurement of the compression strength was carried out with upright moldings along their longest axis after incubation with still wet moldings.

This behavior of the preparations according to the invention was not to be expected because one would assume that a significant increase of the solids content would be achievable only by strong compaction of the particles in the pasty dispersion. This should lead to a corresponding reduction of the oil-filled pores which would greatly impair the displacement of the oil by an aqueous solution (which is necessary for the setting reaction). However, the test results show unexpectedly that, even with a layer thickness of >6 mm, admission of water is reached within <24 h and curing throughout of the moldings occurs without mechanical mixing.

Tests with different surfactants and surfactant combinations proved moreover that the described curing behavior was not at all natural or foreseeable. Even though the exclusive use of anionic surfactants also resulted in very high solids contents in the pastes, no curing throughout of the material did occur however with these preparations even after very long incubation in aqueous solutions. When exclusively using non-ionic surfactants, the pastes after introduction into aqueous solutions exhibited a strong tendency to decompose so that such preparations would be applicable only to a limited extent for clinical use.

Hence, the preparations according to the invention are defined additionally by the fact that they cure throughout without further mixing after introduction into aqueous solutions even in thicker layers (become shape-stable >3 mm within 24 h at 37° C.).

The preparations according to the invention (or special embodiments) are defined furthermore by the fact that they reach a compression strength of >20 MPa after complete curing as a basic material without pore-forming additives and without active agents, without active mixing with an aqueous solution.

Further, it has been found that preparations according to the invention due to the contained combination of several surfactants (preferably at least one non-ionic and one other surfactant, particularly preferred at least one non-ionic and one anionic surfactant) and the stated weight ratio have especially advantageous cohesion properties. It has been demonstrated that the preparations according to the invention have less of a tendency to undergo solid/liquid separation than known pasty cement preparations. The preparations according to the invention are therefore significantly more storage-stable. Preparations according to the invention release with incubation in an aqueous liquid (for example, 0.9% NaCl or simulated body fluid) merely a maximum of about 1 wt. % of filterable particles. Accordingly, moldings made from the pasty preparation maintain their shape very well, even after introduction into an aqueous liquid; particles are hardly released. It is ensured in this way that preparations according to the invention release hardly any particles after introduction into the body (before, during and after the setting reaction). This problem occurs frequently in known cements and is made responsible for cement-caused inflammation reactions.

Preparations according to the invention can be metered very well and are structure-viscous. Even at high solids proportion a reliable metering is thus guaranteed without very high force expenditure. Curing of the preparation after introduction into water or an aqueous solution occurs within a few minutes and can be adjusted in wide ranges by targeted specific dosage of setting accelerator, crystallization seeds, auxiliaries, and by the selection of the components of the mineral cement powder.

Preparations according to the invention with a solids content of more than 85 wt. % exhibit as an especially advantageous handling property a significantly reduced adhesion on gloves and instruments and a very good molding capability.

With the aid of the following embodiments the invention will be explained in more detail without the invention being limited thereby.

The following Examples 1, 2, 4, and 6 were carried out with a mineral cement powder (calcium phosphate cement) of the following composition (values in wt. % relative to the mass of the mineral cement powder):

60 wt. % alpha-tricalcium phosphate, 26 wt. % anhydrous dicalcium phosphate, 10 wt. % calcium carbonate, and 4 wt. % precipitated hydroxylapatite.

In Example 3, a magnesium calcium phosphate cement (MgCPC) of the structure Mg_{2.5}Ca_{0.5}(PO4)₂ was used as a mineral cement powder.

As an organic carrier liquid. Miglyol 812 was used in each case, a saturated part-synthetic medium chain triglyceride. The following surfactants were used in the Examples: anionic surfactant Amphisol A (phosphoric acid monohexadecyl ester), non-ionic surfactant Tween80 (polysorbate 80), and non-ionic surfactant Cremophor ELP (ethoxylated castor oil). Disodium hydrogen phosphate and dipotassium hydrogen phosphate were used as setting accelerator. Grinding occurred in mortars or in ball mills.

### EXAMPLE 1

### Comparative Example Calcium Phosphate Cement Preparation

20 g of CPC cement powder of the abovementioned composition were premixed with 4 g Miglyol 812, 300 mg Na₂HPO₄, 500 mg Tween 80, and 200 mg Amphisol A by hand in a mortar. Afterwards, the mixture was mixed in a 100 ml cup with 10 balls of 10 mm diameter (zirconium dioxide embodiment) three times for 15 min, with 30 min breaks in each case, at 500 revolutions per minute. The result was a homogeneous viscous pasty, slightly sticky preparation. The preparation was filled into a 10 ml syringe and afterwards injected into a beaker with simulated body fluid (SBF) (without cannula). The extruded strand remained substantially intact upon light shaking and cured in less than 60 min to such an extent that it could be removed from the liquid without breaking apart. After about 24 h no change of the compression strength was detectable anymore. A final compression strength is 14 MPa was achieved.

In a laboratory centrifuge 5 g of the afore described preparation were centrifuged in a centrifuge tube at 1,800/min for 15 min. After this time, a thin oil film (low separation tendency of the preparation) formed on the surface of the cement paste. This is an indication that the preparation during storage tends to separate with prolonged storage into a liquid phase and a solid phase.

The preparation after manufacture was filled into a 2 ml disposable syringe (B. Braun Inject® 2 ml Luer solo). The manual discharge from this syringe without attached cannula succeeded with moderate force expenditure. Complete discharge from the syringe was possible. The properties and composition of the comparative preparation are shown in Table 1 (“comparison”).

### EXAMPLE 2

### Preparation According to the Invention with Calcium Phosphate Cement

Preparations with calcium phosphate cements (preparations 1 to 6 in Table 1) were prepared which contained Miglyol 812 as an organic carrier liquid. Two surfactants (Cremophor ELP and Amphisol A) were contained. Dipotassium hydrogen phosphate was used as a setting accelerator. The preparations 1 to 3 were produced without addition of fillers. The preparations 4 to 6 were produced with addition of fillers (dicalcium phosphate anhydride in the preparations 4 and 6, β-tricalcium phosphate in the preparation 5).

The surfactants were mixed homogeneously into the carrier liquid. The calcium phosphate cement and setting accelerator were then added with grinding action (ball mill). The used calcium phosphate cement was comprised of the following components: 60 wt. % α-tricalcium phosphate, 26 wt. % calcium hydrogen phosphate, 10 wt. % calcium carbonate. 4 wt. % precipitated hydroxylapatite.

In addition, the mineral cement powder (calcium phosphate cement) and the setting accelerator were mixed in a ball mill. Afterwards the liquid mixture of surfactants and the organic carrier liquid was added and mixed in the mortar with the powder so that a homogeneous viscous mass is obtained. The mass was ground in a planetary ball mill for a total of five hours (in 500 ml zirconium cup with 8 zirconium balls of an average mass in each case of approx. 110 g). In this context, the rotary speed was increased stepwise to the maximum rotary speed of the planetary ball mill.

For the preparations 4 to 6, a filler (particle size between 63 and 125 μm) as specified was added in each case to the thus prepared mass and was admixed simply by stirring (without grinding). The preparations release easily from the wall of the mixing cup.

In a laboratory centrifuge, in each case 5 g of the different preparations were centrifuged in a centrifuge tube at 1,800/min for 15 min in order to analyze the separation tendency of the preparations. In none of the preparations 1 to 6 according to the invention an oil film formed on the surface of the cement paste. This is an indication that the preparation does not tend to separate into a liquid phase and a solid phase upon prolonged storage.

The preparations were filled in each case into 2 ml disposable syringe. The manual discharge from this syringe without attached cannula succeeded with low to moderate force expenditure. Complete discharge from the syringe was possible. The properties and composition of the different preparation according to the inventions are shown in Table 1.

The compression strength of the obtained moldings was determined with upright moldings of the dimension 6×6×12 mm with a universal testing machine at a feed speed of 1.0 mm/s. The moldings were produced in that the respective preparations according to the invention were introduced into molds open in upward direction that were afterwards introduced into aqueous 0.9% NaCl solution. The measurement of the compression strength of the moldings occurred after four-day incubation in the NaCl solution wherein first the molds filled with the preparations according to the invention were incubated for 24 h at 37° C. and afterwards the removed moldings were incubated for further 72 h at 37° C. and immediately thereafter measured.

TABLE 1

| | Comparison | 1 | 2 | 3 | 4 | 5 | 6 |
|---|---|---|---|---|---|---|---|
| | wt. % | wt. % | wt. % | wt. % | wt. % | wt. % | wt. % |
| Cement powder (CPC) | 80.00 | 83.00 | 82.50 | 83.80 | 62.90 | 62.90 | 54.50 |
| Oil (Miglyol) | 16.00 | 12.30 | 12.20 | 11.10 | 10.00 | 10.00 | 8.67 |
| non-ionic surfactant* | 2.00 | 2.00 | 2.10 | 2.00 | 1.54 | 1.54 | 1.33 |
| setting accelerator** | 1.20 | 2.00 | 2.50 | 2.50 | 1.94 | 1.94 | 1.67 |
| Amphisol A (anionic | 0.80 | 0.70 | 0.70 | 0.60 | 0.62 | 0.62 | 0.54 |
| surfactant) | | | | | | | |
| Filler | 0.00 | 0.00 | 0.00 | 0.00 | 23.00¹ | 23.00² | 33.30¹ |
| Sum wt. % | 100.0 | 100.0 | 100.0 | 100.0 | 100.0 | 100.0 | 100.0 |
| Weight ratio (solid/liquid) | 4.32 | 5.67 | 5.67 | 6.30 | 7.22 | 7.22 | 8.49 |
| Solids content in wt. % | 81.20 | 85.00 | 85.00 | 86.30 | 87.84 | 87.84 | 89.47 |
| Weight proportion | 80.00 | 83.00 | 82.50 | 83.80 | 85.90 | 85.90 | 87.80 |
| cement powder and | | | | | | | |
| filler in wt. % | | | | | | | |
| Initial curing time (min) | 8 | 5 | 4 | 4 | 3 | 3 | 3 |
| Compression strength | 14 | 25 | 25 | 28 | 32 | 33 | 39 |
| after 100 h (MPa) | | | | | | | |
| Discharge force from | moderate | minimal | minimal | minimal | moderate | moderate | n.a. |
| 2.0 ml syringe | | | | | | | |
| Separation tendency | minimal | none | none | none | none | none | none |

*Comparative example Tween 80, preparations 1 to 6 Cremophor ELP
**Comparative example Na₂HPO₄, preparations 1 to 6 K₂HPO₄
¹dicalcium phosphate anhydride,
²β-tricalcium phosphate
n.a. not determined

It is apparent from Table 1 that, in comparison to the comparative preparation, the preparations 1 to 6 all exhibit a significantly improved compression strength after curing and cure faster and the preparations 1 to 6 also do not separate when centrifuged at high rotary speeds which enables a long storage duration. All preparations were dischargeable well and completely from a conventional syringe. After curing no separation of particles was observed when stored in liquid.

### EXAMPLE 3

### Preparation According to the Invention with Magnesium Phosphate Cement for Use in a Single Paste System or Two-Component System with Anhydrous and Water-Containing Paste

Preparations with magnesium calcium phosphate cement (preparations 7 to 11 in Table 2) were produced. As a mineral cement powder, magnesium calcium phosphate cement (MgCPC) of the structure Mg_{2.5}Ca_{0.5}(PO4)₂ was used. It was prepared with CaCO₃ and MgCO₃ in the molar ratio

(Ca+Mg):(PO₄)=3:2

by firing at 1,050° C. and subsequent grinding in a planetary ball mill in cups and by means of grinding balls of zirconium dioxide.

For preparing a pasty preparation, as an organic carrier liquid Miglyol 812 was used, a saturated part-synthetic medium chain triglyceride. The following surfactants were used: anionic surfactant Amphisol A (phosphoric acid monohexadecyl ester), non-ionic surfactant Tween80 (polysorbate 80), and non-ionic surfactant Cremophor ELP (ethoxylated castor oil) elected. The surfactants were admixed homogeneously in the carrier liquid.

The MgCPC powder was mixed and ground with (NH₄)₂HPO₄ and (NH₄)H₂PO₄ in a planetary ball mill with zirconium embodiment with the surfactant-containing organic carrier liquid. For preparing the preparations 8 and 11, CaHPO₄ of an average particle size of 85 μm (sieving fraction) was admixed subsequently to the obtained paste.

For triggering the curing reaction, the obtained pasty preparations were mixed in the ratio given in each case in Table 2 with water (single-paste system: preparations 7, 9, and 10) or an aqueous paste containing 35 wt. % nanocrystalline hydroxylapatite (two-component system: preparations 8 and 11). The preparations 9 to 11 were filled for this purpose into the bigger chamber of a double chamber cartridge and the respective aqueous preparation was filled into the smaller chamber, respectively. Then the contents of both cartridges were forced in parallel through a compulsory mixer and were mixed homogeneously thereby. Setting time, compression strength, separation tendency, and discharge force were determined as in Example I. The compositions and results are shown in Table 2.

TABLE 2

| | 7 | 8 | 9 | 10 | 11 |
|---|---|---|---|---|---|
| | wt. % | wt. % | wt. % | wt. % | wt. % |
| Cement preparation | | | | | |
| Cement powder (MgCPC) | 75.11 | 58.39 | 75.11 | 75.11 | 58.39 |
| Oil (Miglyol) | 10.0 | 11.27 | 10.0 | 10.0 | 11.27 |
| CremophorELP (non-ionic surfactant) | 1.58 | 1.78 | 1.58 | 1.58 | 1.78 |
| (NH₄)₂HPO₄ | 7.49 | 6.08 | 7.49 | 7.49 | 6.08 |
| (NH4)H₂PO₄ | 4.98 | 4.04 | 4.98 | 4.98 | 4.04 |
| Amphisol A (anionic surfactant) | 0.84 | 0.92 | 0.84 | 0.84 | 0.92 |
| Filler CaHPO₄ (Ø 85 μm) | 0.00 | 17.52 | 0.00 | 0.00 | 17.52 |
| Sum wt. % | 100.00 | 100.00 | 100.00 | 100.00 | 100.00 |
| Weight ratio (solid/liquid) | 7.05 | 6.16 | 7.05 | 7.05 | 6.16 |
| Weight proportion cement powder and | 87.58 | 86.03 | 87.58 | 87.58 | 86.03 |
| filler in wt. % | | | | | |
| Aqueous preparation | | | | | |
| Water | 100 | 65 | 100 | 100 | 65 |
| Filler nanocrystalline hydroxylapatite | 0 | 35 | 0 | 0 | 35 |
| Mixing proportion cement | 10:1 | 10:1 | 10:1 | 4:1 | 4:1 |
| preparation:aqueous preparation | | | | | |
| (volume ratio) | | | | | |
| Mixing | Stirring | Stirring | DCS | DCS | DCS |
| Initial setting time (min) | 6 | 4 | 4 | 6 | 4 |
| Compression strength (MPa) | 56³ | 52¹ | 48 ² | 42 ² | 57 ² |
| Discharge force from 2.0 ml syringe (N) | n.a. | n.a. | 85 | 55 | 83 |
| Separation tendency | none | none | None | none | None |

DCS Double chamber syringe with compulsory mixer;
¹ determined after 100 h, ² determined after 6 h,
³ determined after 18 h;
n.a. not determined

All preparations were dischargeable well and completely from a conventional syringe. After curing, no separation of particles was observed upon storage in liquid.

### EXAMPLE 4

### Preparation According to the Invention with Calcium Phosphate Cement for Use in a Two-Component System with Anhydrous and Water Containing Paste

Preparations with calcium phosphate cements (preparations 12 to 14 in Table 3) were prepared which contain Miglyol 812 (a half-synthetic oil) as an organic carrier liquid. Two surfactants (Cremophor ELP and Amphisol A) were contained. As a setting accelerator dipotassium hydrogen phosphate was used. The surfactants were admixed homogeneously into the carrier liquid. Afterwards the calcium phosphate cement and the setting accelerator were added with grinding action (ball mill). The used calcium phosphate cement consisted of 60 wt. % α-tricalcium phosphate, 26 wt. % calcium hydrogen phosphate, 10 wt. % calcium carbonate, 4 wt. % of precipitated hydroxylapatite. Also, a comparative preparation with calcium phosphate cement of the same composition whose formulation is given in Table 3 was produced.

For initiating the curing reaction, the obtained pasty preparations were mixed in a weight ratio of 4:1 with a pasty aqueous solution of 6 wt. % hydroxyethyl starch (aqueous preparation). For this purpose, the preparations (comparative preparation, preparations 12 to 14 according to the invention) were filled into the bigger chamber of a double chamber cartridge and the aqueous preparation into the smaller chamber. Then the contents of both cartridges were pressed in parallel though a compulsory mixer and mixed homogeneously thereby. Curing time, compression strength, separation tendency, and discharge force were determined as in Example 1. The compositions and results are shown in Table 3.

TABLE 3

| | Comparison | 12 | 13 | 14 |
|---|---|---|---|---|
| | wt. % | wt. % | wt. % | wt. % |
| Cement preparation | | | | |
| Cement powder (CPC) | 79.00 | 83.00 | 84.00 | 84.00 |
| Oil (Miglyol) | 17.00 | 13.50 | 12.70 | 12.00 |
| non-ionic surfactant* | 2.00 | 2.00 | 2.10 | 2.00 |
| setting accelerator ** | 1.00 | 0.70 | 0.50 | 1.30 |
| Amphisol A (anionic | 1.00 | 0.80 | 0.70 | 0.70 |
| surfactant) | | | | |
| Sum wt. % | 100.00 | 100.00 | 100.00 | 100.00 |
| Weight ratio (solid/liquid) | 4.00 | 5.13 | 5.45 | 5.80 |
| Solids content in wt. % | 80.00 | 83.70 | 84.50 | 85.30 |
| Weight proportion cement | 79.00 | 83.00 | 84.00 | 84.00 |
| powder (=solids content | | | | |
| without setting accelerator) | | | | |
| in wt. % | | | | |
| Aqueous preparation | | | | |
| Water | 94 | 94 | 94 | 94 |
| Filler hydroxyethyl starch | 6 | 6 | 6 | 6 |
| Mixing ratio cement | 4:1 | 4:1 | 4:1 | 4:1 |
| preparation:aqueous | | | | |
| preparation (volume ratio) | | | | |
| Mixing | DCS | DCS | DCS | DCS |
| Initial curing time (min) | 4.5 | 3.5 | 4.0 | 3.0 |
| Compression strength after | 15.4 | 21.3 | 23.8 | 24.6 |
| 100 h (MPa) | | | | |
| Discharge force from 2.0 ml | 85 | 55 | 58 | 67 |
| syringe (N) | | | | |
| Separation tendency | Minimal | none | none | none |

*Comparative example Tween 80, preparations 12 to 14 CremophorELP
** Comparative example Na₂HPO4, preparations 12 to 14 K₂HPO₄

The preparations 12 to 14 exhibited after curing an improved compression strength in comparison to the known cement preparation (“comparison”). Moreover, a quick initial setting was observed. The preparations 12 to 14 did not separate upon centrifugation at high rotary speeds. All preparations were dischargeable well and completely from a conventional syringe. After curing, no separation of particles was observed upon storage in liquid.

### EXAMPLE 5

### Preparation According to the Invention with Strontium Containing Calcium Phosphate Cement

A calcium ion containing mineral cement powder (strontium containing calcium phosphate cement) of the following composition was produced (values in wt. % relative to the mass of the mineral cement powder):

60 wt. % alpha-tricalcium phosphate, 26 wt. % anhydrous dicalcium phosphate, 10 wt. % strontium carbonate, and 4 wt. % precipitated hydroxylapatite.

In analogy to Example 2, in a planetary ball mill a preparation of the following composition was produced: 82.5 wt. % mineral cement powder, 12.2 wt. % Miglyol 812, 2.1 wt. % Cremophor ELP, 2.5 wt. % KHPO₄, 0.7 wt. % Amphisol A (this corresponds to a solid/liquid weight ratio of 5.67, a solids proportion of 85 wt. %, and a weight proportion of cement powder and filler in the preparation of 82.5 wt. %). Setting time, compression strength, and separation tendency of the thus obtained preparation were determined as in Example 1. The initial setting time was 5 min. The compression strength after 100 h amounted to 38 MPa. No separation tendency and particle release were observed.

### EXAMPLE 6

### Preparation According to the Invention with Calcium Phosphate Cement and Addition of Mineral Glass Fibers

A calcium phosphate cement paste (CPC paste) with the powder composition and the composition of the carrier liquid as in Example 2 was adjusted to a powder:carrier liquid ratio of 85:15. 5 g of a silicic acid fiber with 7.5 μm diameter and approx. 3 mm length was incorporated into 95 g of this CPC paste. Afterwards the CPC paste was still processible well with a spatula. The solids contents of the glass fiber containing paste amounted to 85.75%. The preparation of the test bodies for the determination of the compression strength as well as the determination of the compression strength was carried out as described in Example 2.

FIG. 1 shows the compression strength course across the deformation (stress-strain curve). It can be seen clearly that the deformation behavior of the fiber-reinforced sample (dashed line curve) differs clearly from the sample without reinforcement (solid line curve). While the sample without reinforcement reaches high compression strength, the latter drops catastrophically after surpassing the maximum compression force; the molding is completely destroyed. The reinforced sample shows a significantly different behavior. The maximum compression load is further increased. After having been surpassed, a substantially slower drop occurs and the molding can absorb still considerable forces with further deformation. The molding is deformed, but it remains coherent as such across a wide deformation range.

What is claimed is:

1. Preparation for the manufacture of an implant, preferably a bone implant, comprising:
a) mineral cement powder which contains at least one calcium ion containing and/or at least one magnesium ion containing inorganic compound as a reactive component,
b) at least one organic carrier liquid,
c) at least two surfactants selected from at least two of the groups of the anionic, cationic, amphoteric, and non-ionic surfactants,
d) less than 1 wt. % of water relative to the total mass of the preparation,
wherein the weight ratio of the solids contained in total in the preparation relative to the sum of the weight of the organic carrier liquid and of the at least two surfactants is greater than 5 when the cement powder contains calcium ion containing compounds and contains no magnesium ion containing compounds as a reactive component, with the exception of proportions of magnesium compounds contained as contaminants, or is greater than 6 when the cement powder contains magnesium ion containing compounds or magnesium ion and calcium ion containing compounds as reactive components.

2. Preparation according to claim 1, characterized in that moldings obtainable after curing of the preparation in an aqueous medium have a compression strength of more than 25 MPa.

3. Preparation according to claim 1, characterized in that the preparation has a total solids content of 80 to 95 wt. %.

4. Preparation according to claim 1, wherein at least one setting accelerator is contained, preferably selected from phosphate salts, organic acids or salts of organic acids, preferably potassium ion containing phosphates, wherein the mass of the setting accelerator corresponds to 0.1 to 5% of the mass of the mineral cement powder.

5. Preparation according to claim 1, characterized in that the preparation contains additionally hydrogen phosphates when magnesium ion containing compounds or magnesium ion and calcium ion containing compounds are used.

6. Preparation according to claim 1, wherein at least one non-ionic surfactant is contained, preferably selected from fatty alcohols, ethoxylated fatty alcohols, ethylene oxide/propylene oxide block copolymers, alkylphenol ethoxylates, alkyl polyglucosides, ethoxylated fats and oils, alkanol amides, ethoxylated alkanol amides, polyethylene glycol fatty acid esters, glycol and glycol esters, sorbitan esters, sugar esters, ester/ether surfactants, ethoxylated sorbitan esters, polyglycerin monoesters, and amine oxides.

7. Preparation according to claim 1, wherein at least one anionic surfactant is contained, preferably selected from fatty acids and their salts, esters of fatty acids and their salts, carboxylic acid ethers, alkyl sulfates, alkylether sulfates, alkyl sulfonates, sulfosuccinates, phosphoric acid esters and salts thereof, acylamino acids and salts thereof.

8. Preparation according to claim 1, wherein at least one hydrophilic surfactant with an HLB value of greater than 8 and at least one lipophilic surfactant with an HLB value of less than 5 are contained.

9. Preparation according to claim 1, wherein at least one filler is contained, selected from strontium carbonate, strontium hydrogen phosphate, strontium phosphate, glass ceramics, calcium carbonate, carboxymethyl starch, iron oxides, silicon dioxides, barium sulfate, glycerin stearate, precipitated nanocrystalline hydroxylapatite, calcium deficient hydroxylapatite, and tricalcium phosphate, and mineral or organic fibers.

10. Preparation according to claim 1, wherein at least one anionic and at least one non-ionic surfactant are contained, wherein the total mass of the non-ionic surfactants is preferably at least double the total mass of the anionic surfactants.

11. Preparation according to claim 1 for use for manufacturing as material for treatment of bone defects or for bone augmentation, for anchoring bone implants or for producing implantable active agent carriers.

12. Preparation according to claim 11, characterized in that after curing the preparation has a compression strength of more than 25 MPa.

13. Solid molding obtainable by introducing a preparation according to claim 1 into a water containing preparation or by mixing a preparation according to claim 1 with a water containing preparation.

14. Method for manufacturing a preparation according to claim 1, comprising:
dispersing successively mineral cement powder or individual components of the mineral cement powder by grinding in the organic carrier liquid in presence of the at least two surfactants;
subsequently adding further components of the mineral cement powder and/or fillers, in each case with a particle size of less than 10 μm, with continuous grinding;
afterwards, adding and mixing in coarse fillers with a particle size of more than 50 μm.
